(19) United States
(12) Patent Application Publication
(10) Pub. No.: US 2013/0306067 A1
(43) Pub. Date: Nov. 21, 2013

(54) CEILING-MOUNTED SUPPLY UNIT

(71) Applicant: DRAGER MEDICAL GMBH, Luebeck (DE)

(72) Inventors: Nils CHRISTIANSEN, Luebeck (DE); Dieter SETTGAST, Luebeck (DE); Peter BALTIN, Luebeck (DE); Dhafir JADER, Ratzeburg (DE)

(73) Assignee: DRAGER MEDICAL GmbH, Luebeck (DE)

(21) Appl. No.: 13/837,618

(22) Filed: Mar. 15, 2013

(30) Foreign Application Priority Data
May 15, 2012 (DE) 10 2012 009 639.9

(51) Int. Cl. F16M 13/02; A61M 16/00; A61M 16/10; A61M 16/01

(52) U.S. Cl. CPC F16M 13/027; A61M 16/01; A61M 16/0057; A61M 16/105
USPC 128/203.12; 52/745.21; 52/32

(57) ABSTRACT

A ceiling-mounted supply unit (1) for fastening the medical device (8) on the ceiling-mounted supply unit (1), includes a vertical beam (2), an extension arm (3), to which the vertical beam (2) is fastened especially with a hinge (4), for moving, especially pivoting the vertical beam (2) and electrical and/or pneumatic supply lines with electrical and/or pneumatic connections (21). A medical device (8) is integrated in the vertical beam (2).

### CROSS REFERENCE TO RELATED APPLICATIONS

This application claims the benefit of priority under 35 U.S.C. §119 of German Patent Application DE 10 2012 009 639.9 filed May 15, 2012, the entire contents of which are incorporated herein by reference.

### FIELD OF THE INVENTION

The present invention pertains to a ceiling-mounted supply unit with a vertical beam, an extension arm, to which the vertical beam is fastened, especially with a hinge, for moving, especially pivoting the vertical beam and to a process for manufacturing a ceiling-mounted supply unit.

### BACKGROUND OF THE INVENTION

Ceiling-mounted supply units are used at preoperative and surgical workplaces in hospitals in order to make possible the electric and pneumatic supply of medical devices in the treatment rooms and/or the arrangement of medical devices on the ceiling-mounted supply unit. The operation of the medical devices is simplified hereby, because these are fastened in a mobile manner to the ceiling-mounted supply unit on a ceiling of the building and the floor area is not essentially reduced as a result, and electric and pneumatic connections are mobile. The floor clearance can be increased and crowding of the surrounding area can be reduced as a result in the treatment room.

It is already known that besides pneumatic ports on the ceiling-mounted supply unit, e.g., for oxygen, compressed air and vacuum, as well as an electric connection on the ceiling-mounted supply unit, e.g., at a vertical beam of the ceiling-mounted supply unit, an anesthesia apparatus can be fastened, so that there is an increased space requirement as a result. The anesthesia apparatus is thus fastened to the vertical beam outside a covering of the vertical beam.

DE 87 16 928 U1 shows a ceiling mount for receiving medical devices with an extension arm and with a column and ducted supply lines and with a device mount means, wherein the device mount means is made height-adjustable such that the device mount means receives a medical device being carried by a chassis by gripping under the chassis.

### SUMMARY OF THE INVENTION

An object of the present invention is therefore to provide a ceiling-mounted supply unit and a process for manufacturing a ceiling-mounted supply unit, in which the ceiling-mounted supply unit with the medical device is of a compact design.

This object is accomplished with a ceiling-mounted supply unit for fastening at least one medical device to the ceiling-mounted supply unit, comprising a vertical beam, an extension arm, to which the vertical beam is fastened with a hinge, especially one hinge, for moving, especially pivoting, the vertical beam, of electric and/or pneumatic supply lines, electric and/or pneumatic connections, preferably vertically, wherein at least one medical device is integrated in the vertical beams. The at least one medical device is integrated in the vertical beam, so that the vertical beam requires less space for the medical device as a result. The vertical beam with the medical device can be made substantially more compact as a result and requires less space in a treatment room of the hospital.

The at least one medical device is, in particular, a respirator or anesthesia apparatus. Respirators are used for the artificial respiration of patients, and anesthesia apparatuses are additionally also used for anesthesia of patients during surgery.

In another embodiment, the vertical beam has a holding structure and a covering.

In an additional embodiment, the at least one medical device is arranged at least partially, especially at least 10%, 30%, 50%, 70% or 90% of the volume defined by the medical device, within a space enclosed by the covering structure, and/or the at least one medical device is fastened, especially detachably, to the holding structure and is preferably fastened indirectly with a device-carrying structure to the holding structure and/or part of the housing of the at least one medical device is covered by the covering structure and another part of the housing of the medical device forms the covering structure of the vertical beam, and this other part of the housing is visible and accessible from the outside with the covering structure mounted. The at least one medical device is detachably fastened to the holding structure and can therefore also be replaced in a simple manner for maintenance purposes. A part of the housing of the medical device may also form the covering structure of the vertical beam, so that the housing of the medical device also forms as a result the covering structure of the vertical beam, and the need for covering structure for the vertical beam can be eliminated hereby.

The at least one medical device is preferably split into at least two separate modules, and these at least two modules are fastened separately to the ceiling-mounted supply unit. The at least two modules are fastened or connected here mechanically and/or electrically and/or pneumatically separately to the ceiling-mounted supply unit. There also may be direct electric and/or pneumatic and/or mechanical connections between the modules of the medical device. The mechanical fastening and/or the electrical and/or pneumatic connections of the separate modules can be improved and simplified as a result.

In one variant, the medical device is an anesthesia apparatus and the anesthesia apparatus is split into an electronic module, a fresh gas module and a respiration system module, and these three modules can be replaced at the ceiling-mounted supply unit separately for maintenance purposes. As a result, it is advantageously possible, for example, in case of failure of one component in the fresh gas module, to replace the fresh gas module only, and the other modules can remain, unchanged, at the ceiling-mounted supply unit as a result. The maintenance efforts for the anesthesia apparatus at the ceiling-mounted supply unit can be markedly reduced as a result.

The electronic module preferably comprises a monitor and an electronic unit, and/or the fresh gas module comprises a gas delivery means and/or an anesthetic dispenser with anesthetic storage unit and/or with an anesthetic reflector and/or a feed means for oxygen, and/or the respiration system module comprises a CO₂ absorber and at least one port for a breathing tube.

In another embodiment, the electronic module, fresh gas module and respiration system module are connected to one another, especially detachably, electrically and/or pneumatically with electric and/or pneumatic plug-type connectors, so that when at least one module is removed from the ceiling-mounted supply unit, the electric and/or pneumatic connections are detachable, and when at least one module is fastened to the ceiling-mounted supply unit, the electric and/or pneumatic connections can be connected. The electric and/or pneumatic and/or mechanical connection between the electronic module and/or the fresh gas module and/or the respiration system module may be provided either directly between the modules or indirectly by correspondingly leading supply lines on the rest of the ceiling-mounted supply unit from one module to another module.

In particular, the monitor at the electronic module is connected detachably electrically and mechanically to the electronic unit, so that the monitor can be removed from the electronic unit and can be connected to same and/or the electronic module comprises at least one actuating member, e.g., a button, and./or the monitor as a touch monitor and/or a rotary knob.

Process according to the present invention for manufacturing a ceiling-mounted supply unit, especially a ceiling-mounted supply unit described in this patent application, with the steps of: Providing a vertical beam, an extension arm and a joint, providing electric and/or pneumatic supply lines and connections, connecting the vertical beam, extension arm, hinge, supply lines and connections to the ceiling-mounted supply unit, wherein at least one medical device, especially a respirator or anesthesia apparatus, is integrated in the vertical beam.

In another embodiment, the medical device is fastened at first to a holding structure of the vertical beam, especially detachably, and a covering structure of the vertical beam is then fastened to the holding structure.

In an additional variant, part of the medical device, especially at least 10%, 30%, 50%, 70% or 90% of the volume defined by the medical device, is arranged within a space enclosed by the covering structure after the fastening.

In another variant, the medical device, especially anesthesia apparatus, is split into modules, especially an electronic module, a fresh gas module, and a respiration system module, and the modules are fastened separately to the vertical beam.

In another embodiment, the at least one medical device, especially the modules, are fastened to the vertical beam with a positive-locking and/or non-positive connection, especially a locking, hook-type and/or screw connection, and the at least one medical device, especially the modules, are preferably removed, especially separately, from the vertical beam by loosening the connection. The individual modules can thus be easily connected to the rest of the ceiling-mounted supply unit, so that a module can be easily removed and another module can then again be connected to the ceiling-mounted supply unit with ease to replace a module during maintenance work. Corresponding plug-type connections and counterplug connections are preferably present in this case on the modules and the rest of the ceiling-mounted supply unit, so that a corresponding electric and/or pneumatic connection can also be established automatically between the modules and the rest of the ceiling-mounted supply unit.

In particular, the modules are connected electrically and/or pneumatically and/or the at least one medical device is connected to the electric and/or pneumatic supply lines and/or the at least one medical device is connected to a device-carrying structure indirectly with the holding structure of the vertical beam.

The covering structure and/or the holding structure and/or the device-carrying structure preferably consists, at least partly and especially completely, of metal, e.g., steel or aluminum and/or plastic.

In an additional embodiment, the respirator or anesthesia apparatus has an anesthetic reflector. The anesthetic, e.g., halothane or enflurane, is removed from the expiration gas by means of the anesthetic reflector and the removed anesthetic is subsequently fed to the inspiration gas.

The anesthesia apparatus has an anesthetic dispenser with an anesthetic reservoir in an additional embodiment. Anesthetic is added to the inspiration gas by means of the anesthetic dispenser. The anesthetic is, in general, in the liquid form and the anesthetic is converted in the anesthetic dispenser from the liquid phase into the gaseous phase and thus fed to the patient.

When forming a breathing air circulation system, the expiration gas expired by the patient is fed at least partly to the patient as an inspiration gas. The carbon dioxide is now removed from the expired expiration gas, preferably in the CO₂ absorber, and the inspiration gas to be fed to the patient as a feed means for oxygen is enriched with a mixture of oxygen, nitrous oxide, air and/or xenon in a gas mixer.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side schematic view of a ceiling-mounted supply unit;

FIG. 2 is an exploded view of a vertical beam of the ceiling-mounted supply unit according to FIG. 1;

FIG. 3 is a perspective view of the vertical beam of the ceiling-mounted supply unit according to FIG. 1;

FIG. 4 is a cross sectional view of the vertical beam without holding structure, without device-carrying structure and without fresh gas module, so that only a lateral covering is cut open, the enclosed space is indicated by cross hatching; and

FIG. 5 is a schematic view showing modules with electric and/or pneumatic plug-type connectors to be connected to corresponding electric and pneumatic counterplug connectors on the ceiling-mounted supply unit.

### DESCRIPTION OF THE PREFERRED EMBODIMENTS

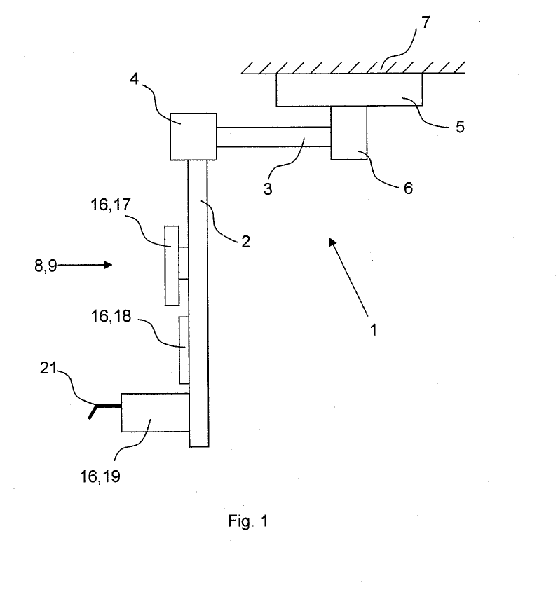

Referring to the drawings in particular, a ceiling-mounted supply unit 1 shown in FIG. 1 is fastened to a ceiling 7 in a building of a hospital for a preoperative or surgical workplace. The ceiling-mounted supply unit 1 comprises an essentially vertically directed vertical beam 2, i.e., one that is directed with a deviation of less than 30°, 20°, 10° or 5° from a vertical plane or axis. The vertical beam 2 is connected to an essentially horizontally directed extension arm 3 by means of a hinge 4 and said extension arm 3 is fastened indirectly to the ceiling 7 with a ceiling joint 6 and with a ceiling fastening part 5. The ceiling-mounted supply unit 1 has pneumatic supply lines, for oxygen, compressed air and vacuum and electric supply lines for current, e.g., with a voltage of 220 V (the supply lines are designated 27—FIG. 5). These electric and pneumatic supply lines 27 end at corresponding electric and pneumatic connections 21, so that medical devices 8 can be supplied as a result electrically and pneumatically in the vicinity of the ceiling-mounted supply unit 1 with the connections 21.

Respirators are used for the artificial respiration of patients and anesthesia apparatuses 9 as a medical device 8 are also used for the anesthesia of patients, besides artificial respiration. The respirator or anesthesia apparatus 9 has a breathing air circuit, i.e., the expiration gas expired by the patient is reused as an inspiration gas for rebreathing. The breathing air is sent by a gas delivery means through gas lines into a breathing air circuit system. A first, inspiratory nonreturn valve and a second, inspiratory nonreturn valve are arranged in the gas lines. As a result, an expiration gas line or an expiration tube and an inspiration gas line or an inspiration tube (not shown) are formed, which are connected as breathing tubes to two connections 21 for the inspiration tube and the expiration tube. A Y-piece (not shown), which sends the inspiration gas and the expiration gas to and from a patient to be respirated artificially by means of a patient connector, is connected at the end of the inspiration line and expiration line. A CO₂ absorber absorbs the carbon dioxide contained in the expiration gas. Furthermore, the inspiration gas is enriched with anesthetic with an anesthetic dispenser. In addition, a mixture of oxygen and nitrous oxide is fed into the inspiration gas by means of a gas mixer.

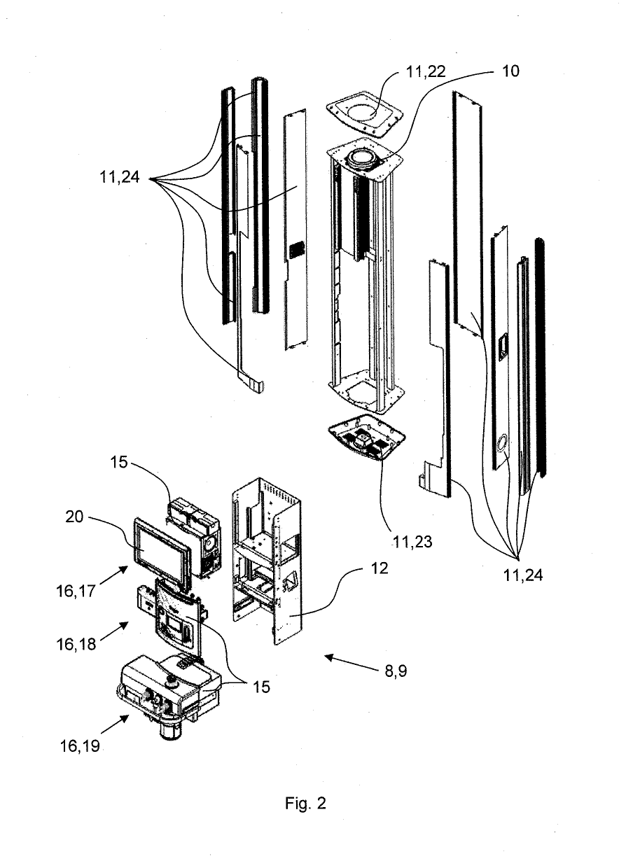

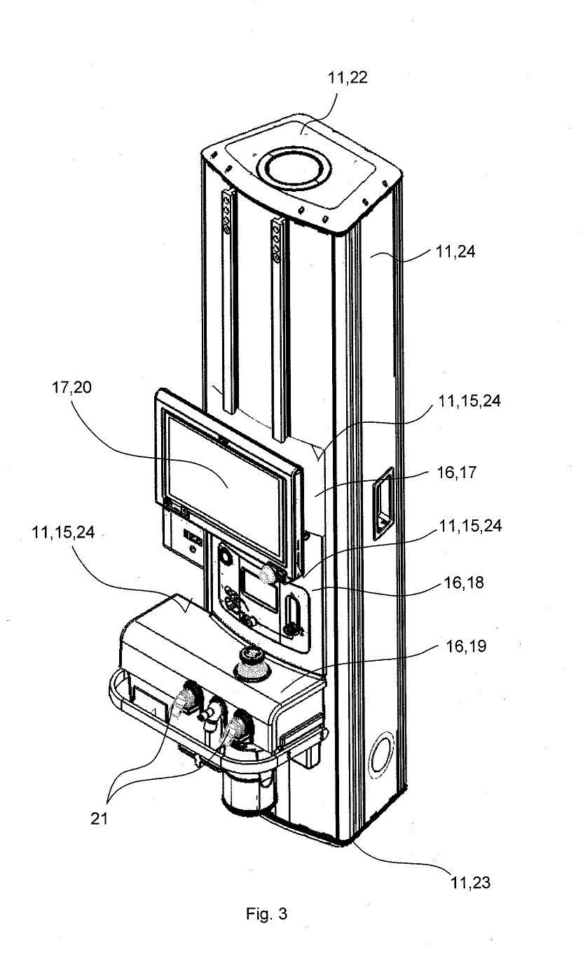

The anesthesia apparatus 9 is integrated in the vertical beam 2 of the ceiling-mounted supply unit 1, FIGS. 2 and 3. The vertical beam 2 has a holding structure 10, which essentially absorbs the mechanical forces to be absorbed and transmits same to the extension arm 3 by means of the joint 4. The holding structure 10, made of metal, is covered by means of a covering structure 11, which comprises an upper covering 22, a lower covering 23 and lateral coverings 24 as well as lateral covering elements 24 (FIGS. 2 and 3). A device-carrying structure 12 is fastened to holding structure 10. The anesthesia apparatus 9 is split into three modules 16, namely, an electronic module 17, a fresh gas module 18 and a respiration system module 19. The electronic module 17 also comprises, besides an electronic unit (not shown, because located inside the housing 15), a monitor 20, and two connections 21 for respiration tubes are present on the fresh gas module 18. Fresh gas module 18 comprises a gas delivery means for the artificial respiration of the patient, an anesthetic dispenser (not shown), as well as a feed means for oxygen. The oxygen is fed to the fresh gas module 18 through corresponding supply lines of the ceiling-mounted supply unit 1. The respiration system module 19 comprises a CO₂ absorber. Each of the three modules 16 has a housing 15, e.g., made of plastic.

The three modules 16 are mechanically, electrically and pneumatically connected to the ceiling-mounted supply unit 1 for the electric and pneumatic supply of the anesthesia apparatus 9 and for mechanical fastening. The modules 16 have corresponding electric and pneumatic plug-type connectors 25, which are connected to corresponding electric and pneumatic counterplug connectors 26 on the vertical beam 2 or at another location on the ceiling-mounted supply unit 1 (FIG. 5). Corresponding electric and/or pneumatic plug-type connectors as well as counterplug connectors may also be formed directly between the modules 16 for the direct electric and pneumatic connection of the modules 16 to one another.

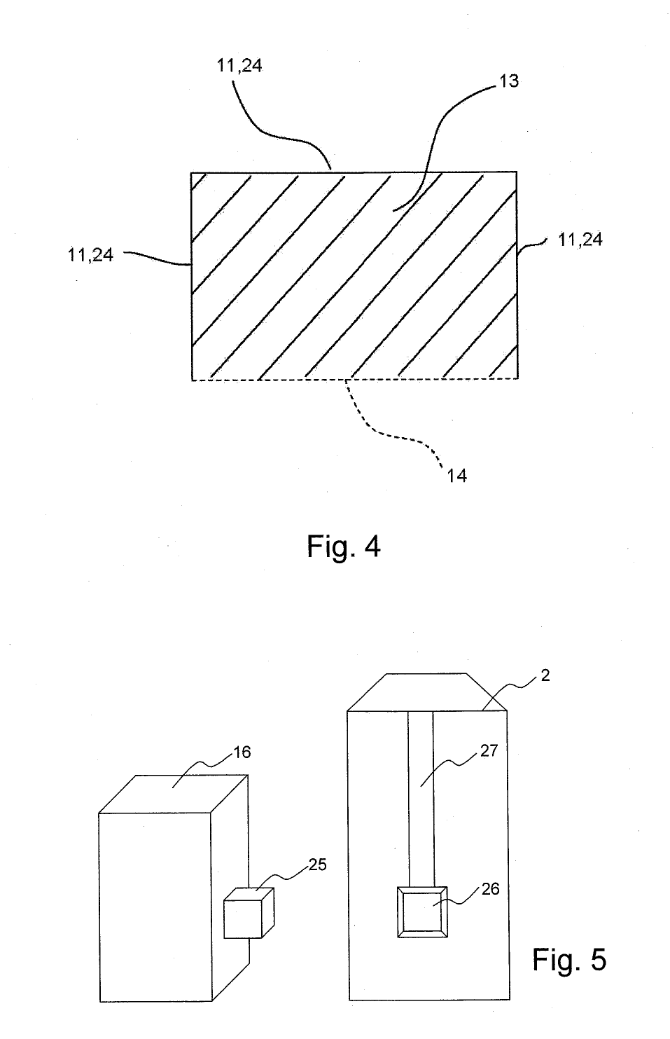

An essential portion of the modules 16 is arranged within a space 13 that is enclosed by the covering structure 11, FIG. 4. According to the sectional view in FIG. 4, covering structure 11 also has an opening 14, and the modules 16 of the anesthesia apparatus 9 are arranged at these openings 14. As a result, the housing 15 of a module 16 also forms a covering structure 11 of the vertical beam 2. According to the view in FIG. 3, for example, a front wall of the housing 15 of the fresh gas module 18 thus also forms, for example, the covering structure 11 of the vertical beam 2. Based on this integration or partial or complete arrangement of the modules 16 of the anesthesia apparatus 9 within the space 13, the vertical beam 2 thus also has a compact design despite the anesthesia apparatus 9 being arranged at the vertical beam 2, because the space 13 enclosed by the covering structure 11 can be used for the accommodation and arrangement of the modules 16 of the anesthesia apparatus 9. The splitting of the anesthesia apparatus 9 into the three modules 16 makes it, furthermore, advantageously possible to have to replace a single module 16 only in case of maintenance work or a failure of individual components in the modules 16. If, for example, the gas delivery means in the fresh gas module 18 fails because of damage to an electric motor, only the fresh gas module 18 needs to be removed from the ceiling-mounted supply unit 1 and a new fresh gas module 18 will subsequently need to be fastened to the ceiling-mounted supply unit 1. The modules 16 are detachably connected mechanically, electrically and pneumatically to the ceiling-mounted supply unit 1 and/or the other modules 16 by means of corresponding plug-type connectors, so that the individual modules 16 can be replaced in a simple manner.

On the whole, essential advantages are associated with the ceiling-mounted supply unit 1 according to the present invention. The anesthesia apparatus 9 is integrated within the vertical beam 2, so that the anesthesia apparatus 9 requires little space at the vertical beam 2 as a result. Based on the splitting of the anesthesia apparatus 9 into three modules 9, the maintenance effort and maintenance costs can be markedly reduced, because only one module 16 rather than the entire anesthesia apparatus 9 needs to be replaced in case of failure of an individual component of the anesthesia apparatus 9.

While specific embodiments of the invention have been shown and described in detail to illustrate the application of the principles of the invention, it will be understood that the invention may be embodied otherwise without departing from such principles.

LIST OF REFERENCE NUMBERS

| | |
|---|---|
| 1 | Ceiling-mounted supply unit |
| 2 | Vertical beam |
| 3 | Extension arm |
| 4 | Hinge |
| 5 | Ceiling fastening part |
| 6 | Ceiling joint |
| 7 | Ceiling |
| 8 | Medical device |
| 9 | Anesthesia apparatus |
| 10 | Holding structure |
| 11 | Covering |
| 12 | Device-carrying structure |
| 13 | Space, enclosed by covering |
| 14 | Opening in covering |
| 15 | Housing of anesthesia apparatus |
| 16 | Module |
| 17 | Electronic module |
| 18 | Fresh gas module |
| 19 | Respiration system module |
| 20 | Monitor |
| 21 | Connection for a breathing tube |
| 22 | Upper covering |
| 23 | Lower covering |
| 24 | Lateral covering |
| 25 | electric and/or pneumatic plug-type connectors |
| 26 | electric and/or pneumatic plug-type connectors |
| 27 | electric and pneumatic supply lines |

What is claimed is:

1. A ceiling-mounted supply unit for fastening at least one medical device on a ceiling-mounted supply unit, the ceiling-mounted supply unit comprising:
a vertical beam;
an extension arm to which the vertical beam is fastened with a hinge, whereby the vertical beam pivots relative to the extension arm;
at least one of electric supply lines and electric connections and pneumatic supply lines and pneumatic connections; and
a medical device integrated in the vertical beam.

2. A ceiling-mounted supply unit in accordance with claim 1, wherein the medical device is a respirator or anesthesia apparatus.

3. A ceiling-mounted supply unit in accordance with claim 1, wherein the vertical beam comprises a holding structure and a covering structure.

4. A ceiling-mounted supply unit in accordance with claim 3, wherein at least one of:
the medical device is arranged such that at least 10% of the medical device is within a space enclosed by the covering structure; and
the medical device is detachably fastened to the holding structure indirectly with a device-carrying structure; and
the medical device includes a housing and a part of the housing of the medical device is covered by the covering structure and another part of the housing of the medical device forms the covering and the another part of the housing is visible and accessible from an outside with the covering structure mounted.

5. A ceiling-mounted supply unit in accordance with claim 1, wherein the medical device is split into at least two separate modules and these at least two modules are fastened separately to the ceiling-mounted supply unit.

6. A ceiling-mounted supply unit in accordance with claim 5, wherein the medical device comprises an anesthesia apparatus split into an electronic module, a fresh gas module and a respiration system module and the electronic module, the fresh gas module and the respiration system module comprise the at least two separate modules.

7. A ceiling-mounted supply unit in accordance with claim 6, wherein at least one of:
the electronic module comprises a monitor and an electronic unit; and
the fresh gas module comprises a gas delivery means and/or an anesthetic dispenser and/or a feed means for oxygen; and
the respiration system module comprises a CO₂ absorber and a breathing tube connection for a breathing tube.

8. A ceiling-mounted supply unit in accordance with claim 7, wherein the electronic module, the fresh gas module and the respiration system module are electrically and/or pneumatically connected to one another detachably, by means of electric and/or pneumatic plug-type connectors, so that the electrical and/or pneumatic connections are detachable when one of the modules is removed from the ceiling-mounted supply unit and the electrical and/or pneumatic connections can be connected when the module is fastened to the ceiling-mounted supply unit.

9. A ceiling-mounted supply unit in accordance with claim 7, wherein at least one of;
the monitor at the electronic module is detachably connected to the electronic unit electrically and mechanically, so that the monitor can be removed from the electronic unit and can be connected to same; and
the electronic module comprises an actuating member including at least one of an actuator element and/or the monitor as a touch actuator monitor and/or a rotary knob.

10. A process for manufacturing a ceiling-mounted supply unit, the process comprising the steps of:
providing a vertical beam, an extension arm by a hinge;
providing at least one of electrical supply lines and connections and pneumatic supply lines and connections;
connecting the vertical beam, the extension arm, the joint, the supply lines and the connections to the ceiling-mounted supply unit, wherein the medical device is integrated in the vertical beam.

11. A process in accordance with claim 10, wherein the medical device is a respirator or anesthesia apparatus.

12. A process in accordance with claim 10, wherein the medical device is fastened detachably to a holding structure of the vertical beam and a covering of the vertical beam is subsequently fastened to the holding structure.

13. A process in accordance with claim 12, wherein at least 10% of the volume defined by the medical device is arranged within a space enclosed by the covering after fastening.

14. A process in accordance with claim 10, wherein the medical device is split into modules including an electronic module, a fresh gas module and a respiration system module, and the modules are fastened separately to the vertical beam.

15. A process in accordance with claim 14, further comprising connectors wherein the medical device modules are fastened with the connectors to form a positive-locking and/or non-positive connection wherein the medical device modules are removed from the vertical beam separately, by severing the connection.

16. A process in accordance with claim 15, wherein the connectors are each one of a locking, hook-type and/or screw connection to the vertical beam.

17. A process in accordance with claim 14, wherein at least one of:
the modules are connected electrically and/or pneumatically to the electrical supply lines and connections and pneumatic supply lines and connections; and
the medical device is connected to the electric and/or pneumatic supply lines; and
the medical device is indirectly connected to a holding structure of the vertical beam with a device-carrying structure.

18. A ceiling-mounted supply unit comprising:
a vertical beam comprising a holding structure, a covering structure and a medical device;
a hinge;
an extension arm connected to the hinge, the vertical beam pivoting relative to the extension arm via the hinge; and
electric supply lines and pneumatic supply lines connected to the vertical beam.

19. A ceiling-mounted supply unit in accordance with claim 18, wherein the medical device comprises modules fastened separately to one of the holding structure and the covering structure.

20. A ceiling-mounted supply unit in accordance with claim 19, wherein the medical device comprises an anesthesia apparatus split into an electronic module, a fresh gas module and a respiration system module and the electronic module, the fresh gas module and the respiration system module comprise the at least two separate modules, wherein at least one of:
the electronic module comprises a monitor and an electronic unit; and
the fresh gas module comprises a gas delivery means and/or an anesthetic dispenser and/or a feed means for oxygen; and
the respiration system module comprises a CO₂ absorber and a breathing tube connection for a breathing tube.
